(19) United States
(12) Patent Application Publication
(10) Pub. No.: US 2022/0257765 A1
(43) Pub. Date: Aug. 18, 2022

(54) TARGET-SPECIFIC CONJUGATE AND USE THEREFOR

(71) Applicant: NATIONAL UNIVERSITY CORPORATION TOKAI NATIONAL HIGHER EDUCATION AND RESEARCH SYSTEM, Nagoya-shi, Aichi (JP)

(72) Inventors: Kazuhide SATO, Nagoya-shi, Aichi (JP); Kazuomi TAKAHASHI, Nagoya-shi, Aichi (JP)

(21) Appl. No.: 17/435,455

(22) PCT Filed: Mar. 2, 2020
(86) PCT No.: PCT/JP2020/008751

§ 371 Date: Sep. 1, 2021

(30) Foreign Application Priority Data
Mar. 5, 2019 (JP) 2019-039986

(51) Int. Cl. A61K 41/00; A61K 47/68; A61P 35/00

(52) U.S. Cl. CPC A61K 41/0071; A61P 35/00; A61K 47/6851

(57) ABSTRACT

An object is to provide a novel therapeutic strategy that solves the problems involved in antibody drug conjugates (ADCs), and that can provide high therapeutic effects. Provided is a photodynamic pharmacotherapy using a target-specific conjugate containing a molecule specifically bindable to a target molecule, a drug, and a substance sensitive to near-infrared light, wherein the drug and the substance sensitive to near-infrared light are linked to the specifically bindable molecule.

### TECHNICAL FIELD

The present invention relates to a target-specific conjugate and the use of the target-specific conjugate. More specifically, the present invention relates to a target-specific conjugate that specifically binds to a target and that locally exerts an action or effect in response to the irradiation of near-infrared light, and relates to a therapeutic method using the target-specific conjugate. The present application claims priority based on Japanese Patent Application No. 2019-39986, filed on Mar. 5, 2019, the entire disclosure of which is incorporated herein by reference.

### BACKGROUND ART

Antibody drug conjugates (ADCs) are promising biopharmaceuticals composed of an antibody and a small-molecule drug linked to via a linker (see NPL 1). ADCs are believed to be usable in wider therapeutic areas and have greater efficacy than conventional chemotherapeutic drugs due to their efficient and specific delivery of a drug to tumor cells expressing an antigen. Whereas ADCs have shown excellent results in some clinical trials, the development of ADCs has been delayed by some drawbacks such as a lack of antitumor effects, side effects caused by drugs bound to antibodies, and inability to administer sufficient drug doses due to side effects. In particular, intratumoral heterogeneity of the expression of a target cancer antigen within the same tumor and the difficulty of delivering a drug deep into a tumor are partially responsible for the resistance to therapy in solid cancer (see NPL 2). Thus, the antibodies can bind only to a limited area of a tumor, and ADCs cannot reach deep enough into a tumor, thus failing to demonstrate an expected efficacy. This appears to result in treatment failure and Induction of drug resistance.

### CITATION LIST

### Patent Literature

PTL 1: US Patent Application Publication No. 2014/0120119
PTL 2: US Patent Application Publication No. 2018/0150405
PTL 3: US Patent Application Publication No. 2014/0120119

### Non-Patent Literature

NPL 1: Beck A et al., Nat Rev Drug Discov. 2017 May; 16(5): 315-337.
NPL 2: Heppner, G H, Cancer Res. 1984; 44: 2259-2265.
NPL 3: Mitunaga M et al., Nat Med. 2011; 1685-1691.
NPL 4: Ogawa, M et al., Oncotarget. 2017, 8(6), 10425-10436.
NPL 5: Sato K et al., ACS Cent. Sci. 2018, 4(11), pp 1559-1569.

### SUMMARY OF INVENTION

### Technical Problem

Although being promising drugs, ADCs have problems such as side effects and insufficient efficacy. Thus, an object of the present invention is to provide a novel therapeutic strategy that solves the problems faced by ADCs and that also demonstrates high therapeutic effects.

### Solution to Problem

In the course of research, the inventors focused on near-infrared photoimmunotherapy (NIR-PIT), taking into consideration the characteristics and unique effects of ADCs. NIR-PIT is a novel cancer phototherapy that binds a photosensitive substance (e.g., IRdye700DX (IR700)) to an antibody (or other ligand, peptide, minibody, diabody, scFv etc.) and targets a specific cell surface molecule. An antibody-IR700 conjugate, containing IR700 as a photosensitive substance, binds to a target cell through an antigen-antibody reaction, and then irradiating the target cell with near-infrared light at 690 nm (excitation wavelength of IR700) induces target-selective necrotic cell death. However, this does not cause toxicity to the adjacent non-target cells (see NPL 3). Additionally, NIR-PIT showed that adjacent dendritic cells respond to cancer antigens exposed due to cell death and then mature, thereby activating the cancer immunity of the host (see NPL 4). Studies in recent years have also revealed that the mechanism underlying the antitumor effects of NIR-PIT is a photochemical reaction that is completely different from that of existing antitumor therapies (see NPL 5). As noted above, NIR-PIT is an innovative technology capable of providing antitumor effects by a mechanism different from that of the conventional techniques, and has already been in international phase III clinical trials. Thus, NIR-PIT is expected to have wide applications as a new technology in the future clinical treatment settings. PTL 1 to 3 propose using NIR-PIT in the treatment of tumors.

However, a cancer tissue is not homogeneous (heterogeneous), and is rather an inhomogeneous assembly of cancer cells. The expression of cancer antigen is thought to vary from cell to cell. As mentioned above, NIR-PIT is effective against cancer cells expressing many target cancer antigens (target cancer cells); however, due to the inability to reach or bind to cancer cells expressing fewer target cancer antigens (non-target cancer cells) even within the same cancer tissue, NIR-PIT may not produce an antitumor effect.

The inventors thought that combining ADCs with NIR-PIT would bring an innovative therapeutic strategy that could solve the problems of ADCs while enhancing the effects of NIR-PIT, and decided to study the effectiveness. As shown in the Examples below, ADC-IR700 conjugates that were prepared by conjugating ADC and IR700 and that were subjected to NIR-PIT had higher antitumor effects than those treated with ADC alone both in vitro and in vivo. The mechanism appears to be as follows. NIR-PIT first causes the cell death of target cancer cells to destroy a tumor, and allows a drug (payload) to leave the ADC at the same time. The released payload is widely diffused within the destroyed tumor, and acts not only on the target cancer cells but also on non-target cancer cells, resulting in a strong antiproliferative effect on the tumor. The effects of ADCs and NIR-PIT on non-target cells have not been reported before, and this is a novel, innovative technology of non-targeted therapies using light rays. Although there was a concern particularly about whether the release and diffusion of a payload from an ADC could be successfully achieved, the effects greatly exceeded the inventor's expectations.

The following subject matter is provided mainly on the basis of the above achievements and studies.

[1] A target-specific conjugate comprising
a molecule specifically bindable to a target molecule,
a drug, and
a substance sensitive to near-infrared light, wherein the drug and the substance sensitive to near-infrared light are linked to the specifically bindable molecule.
   [2] The target-specific conjugate according to [1], wherein the specifically bindable molecule is an antibody or an antigen-binding antibody fragment.
   [3] The target-specific conjugate according to [1] or [2], wherein the target molecule is a cell surface protein.
   [4] The target-specific conjugate according to [3], wherein the cell surface protein is a tumor-specific protein.
   [5] The target-specific conjugate according to [4], wherein the tumor-specific protein is HER1, HER2, HER3, CD3, CD19, CD20, CD25, CD26, CD33, CD44, CD52, PDL-1, CTLA-4, EpCAM, GD2, VEGFR, VEGFR2, CCR4, PMSA, mesothelin, GPC3, CEA, MUC1, c-KIT, DLL-3, PDPN, GPR85, GPR78, Cadherin3, Trop-2, B7-H3, or an ephrin receptor.
   [6] The target-specific conjugate according to any one of [1] to [5], wherein the drug is a cell-damaging drug.
   [7] The target-specific conjugate according to [6], wherein the cell-damaging drug is one, or two or more drugs selected from the group consisting of alkylating agents, platinum-based drugs, antimetabolites, antitumor antibiotics, microtubule polymerization inhibitors, microtubule depolymerization inhibitors, topoisomerase inhibitors, vegetable alkaloids, hormonal agents, and bacteria-derived toxins.
   [8] The target-specific conjugate according to [4] or [5], wherein the drug is an anticancer agent.
   [9] The target-specific conjugate according to any one of [1] to [8], wherein the substance sensitive to near-infrared light is a phthalocyanine dye.
   [10] The target-specific conjugate according to [9], wherein the phthalocyanine dye is IR700.
   [11] A pharmaceutical composition comprising the target-specific conjugate of any one of [1] to [10].
   [12] The pharmaceutical composition according to [11], which is for use in the treatment or prevention of a cancer.
   [13] The pharmaceutical composition according to [12], wherein the cancer is non-small cell lung cancer, small-cell lung cancer, breast cancer, stomach cancer, large intestine cancer, kidney cancer, head and neck cancer, malignant melanoma, Hodgkin's lymphoma, B-cell non-Hodgkin's lymphoma, mantle cell lymphoma, chronic lymphocytic leukemia, Philadelphia chromosome-positive acute lymphocytic leukemia, multiple myeloma, adult T-cell leukemia, peripheral T-cell lymphoma, cutaneous T-cell lymphoma, neuroblastoma, bladder cancer, ureteral cancer, angiosarcoma, rectal cancer, anus cancer, small intestine cancer, duodenal cancer, pancreas cancer, bile duct cancer, liver cancer, gallbladder cancer, esophageal cancer, GIST, malignant mesothelioma, thymic tumor, oral cancer, or brain tumor.
   [14] A therapeutic method comprising the following steps (1) and (2):
   (1) administering the pharmaceutical composition of any one of claims 11 to 13 to a treatment target to bind the target-specific conjugate to a target cell, and
   (2) irradiating the target cell with near-infrared light.
   [15] The therapeutic method according to [14], wherein the near-infrared light has a wavelength of 660 to 740 nm.
   [16] The therapeutic method according to [14], wherein the near-infrared light has a wavelength of 670 to 720 nm.
   [17] The therapeutic method according to any one of [14] to [16], wherein an irradiation dose of the near-infrared light is 1 J cm⁻² or more.
   [18] The therapeutic method according to any one of [14] to [16], wherein an irradiation dose of the near-infrared light is 2 J cm⁻² to 500 J cm⁻²,
   [19] The therapeutic method according to any one of [14] to [16], wherein an irradiation dose of the near-infrared light is 5 J cm⁻² to 300 J cm⁻²,
   [20] The therapeutic method according to any one of [14] to [19], wherein after the irradiation with the near-infrared light induces necrotic cell death, the drug linked to the target-specific conjugate is diffused to cause damage to surrounding cells.

### BRIEF DESCRIPTION OF DRAWINGS

FIG. 1: The confirmation of quality of T-DM1 (Trastuzumab+N2′-deacetyl-N2′-maytansine)-IR700. The results of SDS-PAG (left: protein staining, right: fluorescence measurement).

FIG. 2: The confirmation of quality of T-DM1-IR700. Fluorescence measurement of 3T3/HER2 (left) and fluorescence measurement of MDA-MB-468 (right). Cont: control, A: T-DM1-IR700 (10 μg/ml), B: T-DM1-binding inhibition (Trastuzumab (Tra) (100 μg/ml)+T-DM1-IR700.

FIG. 3: Evaluation of cell proliferation inhibition activity. The survival rate of 3T3/HER2 (left) and the survival rate of MDA-MB-468 (right). Data are presented as average±standard deviation (SD) (n=4).

FIG. 4: An experimental method of in vitro NIR-PIT.

FIG. 5: The results of in vitro NIR-PIT. Data are presented as average±standard error of the mean (SEM) (n=4). **p<0.0001, and *p<0.01 in Student's t-test

FIG. 6: An experimental method of in vivo NIR-PIT.

FIG. 7: The results of in vivo NIR-PIT.

### DESCRIPTION OF EMBODIMENTS

### 1. Target-Specific Conjugate

The first aspect of the present invention relates to a “target-specific conjugate,” which is an assembly specifically bindable to its target (the target of attack). The target-specific conjugate according to the present invention has a structure such that a drug and a substance sensitive to near-infrared light are linked to a molecule specifically bindable to its target surface molecule (“specifically bindable molecule” below).

The target of attack of the target-specific conjugate according to the present invention is cells or pathogens (viruses, bacteria, parasites, etc.) involved in a disease or pathological condition. The “cells involved in a disease or pathological condition” are those that cause a disease or pathological condition, or those necessary for the formation (configuration), maintenance, progression, or exacerbation of a disease or pathological condition (including subservient cells). Examples of such cells include tumor cells, cancer cells, immune cells (T cells such as helper T cells, cytotoxic T cells, and regulatory T cells, B cells such as plasma cells, memory B cells, and naive B cells, natural killer (NK) cells, monocytes, macrophages, dendritic cells, lymphoblasts, and lymphocyte progenitors), virus-infected cells, bacteria-infected cells, and parasite-infected cells.

Preferable examples of cells to be attacked by the target-specific conjugate according to the present invention are cells existing in the microenvironment of a lesion, such as tumor cells, cancer cells, lymphocytes, stromal cells (fibroblasts, immune cells, pericytes, endothelial cells, inflammatory cells (neutrophils, eosinophils, basophils, lymphocytes, macrophages, mast cells etc.)), various cells infected by pathogens (viruses, bacteria, parasites, etc.), various cells infected with pathogens (viruses, bacteria, parasites, etc.) (airway epithelial cells, intestinal epithelial cells, hepatocytes, various nerve cells, various immune cells, blood cells, etc.), and pathogen cells.

The phrase “specifically bindable molecule” refers to a molecule that exhibits specific binding properties for a molecule expressed in the target of attack (target molecule) of the target-specific conjugate. Typical examples of target molecules are those that are expressed on the surface of the target of attack and that are externally presented or exposed, i.e., surface molecules (e.g., surface proteins). The target molecules include peptides, proteins, lipids, polysaccharides, proteoglycans, lipopolysaccharides, nucleic acids, etc. Specific examples include receptors, surface antigens (e.g., tumor-specific proteins (also called “tumor antigens”) that are highly or specifically expressed on the cell surface of tumor cells or cancer cells, and surface proteins that are highly or specifically expressed in specific immune cells), and pathogen-derived molecules that are highly or specifically expressed on the cell surface of pathogen-infected cells such as virus-infected cells, bacteria-infected cells, and parasite-infected cells. Specific examples of surface molecules include HER1/EGFR, HER2/ERBB2, HER3, CD3, CD11, CD18, CD19, CD20, CD25, CD26, CD30, CD33, CD44, CD52, CD133, CD206, CEA (carcinoembryonic antigen), CA125 (cancer antigen 125), AFP (alpha-fetoprotein), TAG72, caprin-1, mesothelin, PD-1, PDL-1, CTLA-4, IL-2 receptor, IL-6 receptor, VEGF (vascular endothelial growth factor), EpCAM, EphA2, GPC3 (glypican-3), gpA33, mucin, CAIX, PSMA, MART-1/Melan-A, Mage-1, Mage-3, gp100, ganglioside (e.g., GD2, GD3, GM1, and GM2), VEGFR, VEGFR2, ERBB3, IGF1R, EPHA3, TRAILR1, TRAILR2, RANKL, FAP, tenascins, AFP, gp72, MUC, nuC242, PEM antigen, ephrin receptor, HGF receptor, CXCR4, bombesin receptor, SK-1, PGR (progesterone receptor), PSA (prostate-specific antigen), PSCA (prostate stem cell antigen), PSMA (prostate-specific membrane antigen), NY-ESO-1, mutant ras, mutant p53, HPV 16/18, HVP E6/E7, Lewis Y antigen, CCR4, SLAMF7, PMSA, c-KIT, DLL-3, PDPN, GPR85, GPR78, Cadherin3, Claudin, Trop-2, and B7-H3.

The conjugate is given directionality toward the target due to the use of the specifically bindable molecule. The phrase “specifically bindable” refers to the ability or properties to bind to a target with a clearly and significantly higher affinity, such as binding in an antigen-antibody reaction, than binding to a non-target. In a preferable embodiment, the specifically bindable molecule does not have a substantial binding force to non-targets.

Examples of the specifically bindable molecule include antibodies, antigen-binding antibody fragments, ligands of receptors, peptides, polypeptides, virus particles, virus capsids, oligosaccharides, polysaccharides, nucleic acids (artificial nucleic acids such as DNA, RNA, LNA (locked nucleic acid), and BNA (bridged nucleic acid)), peptide nucleic acids, exosomes, and nanoparticles.

In a preferable embodiment, the specifically bindable molecule is an antibody or an antigen-binding antibody fragment of the antibody. The term “antibody” refers to a protein molecule that specifically recognizes the epitope of an antigen and binds to the antigen. The term “antibody” includes various forms of antibodies, such as chimeric antibodies, humanized antibodies, and human antibodies. The phrase “chimeric antibodies” refer to those having variable regions derived from a non-human animal species (e.g., mice), while having the other constant regions replaced with human-derived immunoglobulins. The phrase “humanized antibodies” refer to those having the complementarity-determining regions (CDR) of the variable regions derived from a non-human animal species (typically mice) while having the other framework regions (FR) derived from humans. The phrase “human antibodies” (also known as “fully human antibodies”) refers to antibodies that contain CDRs derived from human immunoglobulins and human FRs. For example, human antibodies are produced by using transgenic mice that have a human antibody gene introduced.

The phrase “antigen-binding antibody fragment” (abbreviated as “antibody fragment” below) contrasts to a complete (intact) antibody, and contains the portions of an antibody necessary for antigen-binding properties and retains the ability to bind to an antigen. Examples of antibody fragments include scFv, Fab, Fab′, F(ab′)2, scFv-CH3 (minibody), scFv-Fc, diabody, and single-chain diabody (scDb).

Multispecific antibodies that can bind to two or more antigens, such as bispecific antibodies, are also usable. Examples of multispecific antibodies include diabodies, scDb, CrossMAb (Roche), DuoBody (registered trademark, Genmab), Two-in-One antibodies, DutaMab (Creative Biolabs), DVD-Ig™ (Dual-Variable Domain Immunoglobulin) (Abbott), ART-Ig (registered trademark, Chugai Pharmaceutical Co., Ltd.), BiTE (registered trademark, Amgen), and DART (Dual-Affinity Re-targeting Antibody) (registered trademark, Amgen).

Antibodies or antibody fragments can be produced as specifically bindable molecules by a known method. Examples of usable methods include an immunological method (e.g., a hybridoma method), a phage display method, a ribosome display method, a method using genetically modified mice, immortalization of human antibody-producing cells by EB virus, and a fusion partner method using a SPYMEG technique (WO2007/119808A). Various antibodies specific for tumor antigens usable as antibody drugs have also been developed, and these antibodies may also be used as specifically bindable molecules. Specifically, an existing or to-be-developed desired antibody may be purchased and used as a specifically bindable molecule. The following shows specific examples of antibodies usable as specifically bindable molecules, with the generic name (trade name) of the antibody drugs, the target molecule, and the main application.

Rituximab (Rituxan (registered trademark)); CD20; B-cell non-Hodgkin's lymphoma, MCL (mantle cell lymphoma)
Trastuzumab (Herceptin (registered trademark)); HER2; breast cancer, stomach cancer
Alemtuzumab (Campath (registered trademark)); CD52; CLL (chronic lymphocytic leukemia)
Cetuximab (Erbitux (registered trademark)); EGFR; large intestine cancer, head and neck cancer
Panitumumab (Vectibix (registered trademark)); EGFR; large intestine cancer
Ofatumumab (Arzerra (registered trademark)); CD20; CLL
Denosumab (Ranmark (registered trademark)); RANKL; bone lesions due to multiple myeloma, bone lesions due to solid cancer bone metastasis, bone-related event prevention, and giant cell tumor of bone
Ipilimumab (Yervoy (registered trademark)); CTLA-4; malignant melanoma
Mogamulizumab (Poteligeo (registered trademark)); CCR4; ATL (adult T-cell leukemia), PTCL (peripheral T-cell lymphoma), CTCL (skin T-cell lymphoma)
Pertuzumab (Perjeta (registered trademark)); HER2; breast cancer
Obinutuzumab (Gazyva (registered trademark)); CD20; CLL
Ramucirumab (Cyramza (registered trademark)); VEGFR2; gastric adenocarcinoma and gastroesophageal junction adenocarcinoma, non-small cell lung cancer, large intestine cancer
Nivolumab (Opdivo (registered trademark)); PD-1; malignant melanoma, non-small cell lung cancer, kidney cancer, Hodgkin's lymphoma, head and neck cancer, stomach cancer, small-cell lung cancer
Pembrolizumab (Keytruda (registered trademark)); PD-1; malignant melanoma, non-small cell lung cancer
Blinatumomab (Blincyto (registered trademark)); CD19/CD3; Ph-ALL (Philadelphia chromosome-positive acute lymphocytic leukemia)
Dinutuximab (Unituxin (registered trademark)); GD2; neuroblastoma
Daratumumab (Darzalex (registered trademark)); CD38; multiple myeloma
Necitumumab (Portrazza (registered trademark)); EGFR; non-small cell lung cancer
Elotuzumab (Empliciti (registered trademark)); SLAMF7; multiple myeloma

The specifically bindable molecule has a drug (payload) linked thereto. During a treatment using the target-specific conjugate according to the present invention (e.g., treatment of cancer), the drug is released and comes into contact with or is taken up by the surrounding cells, thereby exerting its medicinal effect (details are described later). Thus, the target-specific conjugate according to the present invention clearly differs from and contrasts to radioimmunotherapeutic drugs that cause damage to surrounding cells due to the radiation emitted from a radioisotope (e.g., ibritumomab tiuxetan) in terms of the structure and action mechanism.

The drug may be a prodrug. Drugs capable of causing damage (i.e., damage-causing drugs) are used. A typical example of such drugs is a cell-damaging drug (cytotoxic drug). The cell-damaging drug is a compound that kills cells (e.g., cancer cells), induces cell death, or reduces the growth rate or survival rate of cells. Examples of cell-damaging drugs include alkylating agents, platinum-based drugs, antimetabolites, antitumor antibiotics, microtubule polymerization inhibitors, microtubule depolymerization inhibitors, topoisomerase inhibitors, vegetable alkaloids, hormonal agents, and bacteria-derived toxins. Examples of alkylating agents include cyclophosphamide, ifosfamide, nitrosourea, dacarbazine, temozolomide, nimustine, busulfan, melphalan, thiotepa, procarbazine, and ranimustine. Examples of platinum-based drugs include cisplatin, nedaplatin, oxaliplatin, and carboplatin. Examples of antimetabolites include enocitabine, carmofur, capecitabine, tegafur, tegafur-uracil, tegafur-gimeracil-oteracil potassium, gemcitabine, cytarabine, cytarabine ocfosfate, nelarabine, fluorouracil, fludarabine, pemetrexed, pentostatin, methotrexate, cladribine, doxifluridine, hydroxycarbamide, and mercaptopurine. Examples of antitumor antibiotics include mitomycin C, doxorubicin, epirubicin, daunorubicin, bleomycin, actinomycin D, aclarubicin, idarubicin, pirarubicin, peplomycin, mitoxantrone, amrubicin, and zinostatin stimalamer. Examples of microtubule polymerization inhibitors include vinblastine, vincristine, and vindesine. Examples of microtubule depolymerization inhibitors include paclitaxel and docetaxel. Examples of topoisomerase inhibitors include irinotecan, nogitecan, etoposide, and sobuzoxane. Maytansinoids and maytansinoid analogs (e.g., emtansine (M4-1)), which are used in ADCs for molecularly targeted drugs targeting cancer, are also another preferable example of cell-damaging drugs.

For targeting tumor cells or cancer cells, anticancer agents (e.g., alkylating agents, platinum-based drugs, antimetabolites, antitumor antibiotics, microtubule polymerization inhibitors, microtubule depolymerization inhibitors, and topoisomerase inhibitors) are typically used as a cell-damaging drug.

Although a single drug is typically used, the use of two or more drugs in combination is not excluded. Specifically, two or more drugs may be linked to the specifically bindable molecule.

Techniques for linking a drug to a specifically bindable molecule (in particular, an antibody) are well known. For example, linkers and spacers are used. For these techniques, the following can be referred to: U.S. Pat. Nos. 7,090,843, 7,091,186, 7,223,837, 7,553,816, 7,659,241, 7,989,598, 8,163,888, 8,198,417, 8,236,319, 8,563,509, 6,214,345, 4,563,304, WO2009/0134976, WO2009/134977, WO2012/177837, Yoshitake et al. (1979) Eur. J. Biochem., 101, 395-399, Carlsson et al., Biochem. J., 173: 723-737 (1978), Controlled Drug Delivery: Fundamentals and Applications, second edition (Drugs and the Pharmaceutical Sciences) by Joseph Robinson, R. W. Baldwin, Monoclonal Antibodies for Cancer Detection and Therapy, Academic Press, 1985, and Thorpe et al., The Preparation and Cytotoxic Properties of Antibody-Toxin Conjugates, Immunol. Rev., vol. 62, 1982, pages 119-58. Examples of linkers or spacers include maleimidecaproyl, maleimidecaproyl-polyethylene 20-glycol (MC(PEG)6-OH), p-aminobenzylcarbamoyl (PAB), lysosomal-enzyme-cleavable linker, valine-citrulline (vc), N-methyl-valine-citrulline, N-succinimidyl 4-(N-maleimidemethyl) cyclohexane-1-carboxylate (SMCC), N-succinimidyl 4-(2-pyridyldithio)butanoate (SPDB), N-succinimidyl 4-(2-pyridyldithio)2-sulfobutanoate (sulfo-SPDB), N-succinimidyl 3-(2-pyridyldithio)propionate (SPDP), N-succinimidyl 4-(2-pyridyldithio)pentanoate (SPP), 2-iminothiolane, and acetylsuccinic anhydride.

In existing ADCs, a drug is usually linked to an antibody by using a conjugation reaction targeting lysine or cysteine. In order to prepare more homogeneous ADCs, the following methods have been proposed: selective bioconjugation reaction due to incorporation of unnatural amino acids, introduction of free cysteine by genetic modification (THIOMAB), a method of performing a conjugation reaction by exposing aldehyde from a sequence containing an N-terminal or free cysteine (SMARTag), and a ligation method using an enzyme. These techniques may also be applied as a means of linking a drug to a specifically bindable molecule.

The number (amount) of drugs linked to the specifically bindable molecule is not limited, and may be, for example, 1 to 10 per specifically bindable molecule (if the specifically bindable molecule is an antibody, drug-to-antibody ratio DAR: 1 to 10), preferably 1 to 8 per specifically bindable molecule (drug-to-antibody ratio DAR: 1 to 8).

The present invention uses the principle of photoimmunotherapy (PIT). Thus, the specifically bindable molecule is linked with a substance sensitive to near-infrared light in addition to the drug. Typically, the substance sensitive to near-infrared light for use is a phthalocyanine dye. The phthalocyanine dye refers to a group of photosensitizer compounds with a phthalocyanine ring. For example, WO2005/099689 and U.S. Pat. No. 7,005,518 can be referred to for the method for synthesizing or using (applying) various phthalocyanine dyes.

A preferable phthalocyanine dye for use has an absorption peak in the near-infrared (NIR) region and strongly absorbs near-infrared rays to emit fluorescence. More specifically, a preferable phthalocyanine dye for use has an absorption peak preferably within the range of 600 nm and 950 nm, more preferably 660 nm and 740 nm, and still more preferably 680 nm and 720 nm.

A particularly preferable phthalocyanine dye is IR700 (IRDye (registered trademark) 700DX). IR700 is commercially available from LI-COR Biosciences. The amino-reactive IR700 is a relatively hydrophilic dye. For example, IR700 can covalently bind (conjugate) to antibodies or other substances through its NHS ester.

The substance sensitive to near-infrared light is directly or indirectly linked to the specifically bindable molecule through a covalent or non-covalent bond. A non-covalent bond can be made, for example, by electrostatic interaction, a van der Waals force, hydrophobic interaction, a n-effect, ionic interaction, a hydrogen bond, or a halogen bond. For an indirect bond, a linker is typically used.

Incidentally, antibody-drug conjugates (ADCs), which are molecularly targeted drugs composed of an antibody linked to a small-molecule drug, have been developed. It is also possible to prepare the target-specific conjugate according to the present invention by using an existing or to-be-developed ADC. Specifically, the target-specific conjugate according to the present invention may be prepared by linking a substance sensitive to near-infrared light to an ADC (optionally with further modifications or alterations). The following shows specific examples of ADCs, with the generic name (trade name), the target molecule, and the main application.

Gemtuzumab ozogamicin (Mylotarg (registered trademark)); CD33; relapsed or refractory AML (acute myeloid leukemia)
Brentuximab vedotin (Adcetris (registered trademark)); CD30; relapsed or refractory Hodgkin's lymphoma, anaplastic large cell lymphoma
Trastuzumab emtansine (Kadcyla (registered trademark)); HER2; breast cancer
Inotuzumab ozogamicin (BESPONSA (registered trademark); CD22; relapsed or refractory precursor B-cell acute lymphoblastic leukemia
Rovalpituzumab tesirine (Rova-T); DLL-3; small-cell lung cancer, large cell neuroendocrine carcinoma
Sacituzumab govitecan (Sacituzumab Govitecan); Trop-2; urothelial cancer, breast cancer

### 2. Pharmaceutical Composition and Use of the Composition

The target-specific conjugate according to the present invention can be formulated to prepare a pharmaceutical composition. In general, a pharmaceutically acceptable carrier (carrier, vehicle) is used for such a formulation. Examples of carriers include water, physiological saline, a balanced salt solution, aqueous dextrose, glycerol, mannitol, lactose, starch, and magnesium stearate. For information on pharmaceutically acceptable carriers and their use, see, for example, Remington's Pharmaceutical Sciences, by E. W. Martin, Mack Publishing Co., Easton, Pa., 19th Edition (1995).

In addition to the carrier, the pharmaceutical composition may contain, for example, a diluent (lactose, sucrose, dicalcium phosphate, carboxymethylcellulose, etc.), an excipient (starch, glucose, lactose, sucrose, gelatin, malt, rice, wheat flour, chalk, silica gel, sodium stearate, glycerol monostearate, talc, sodium chloride, dried skim milk, glycerol, propylene glycol, water, ethanol, etc.), a lubricant (magnesium stearate, calcium stearate, talc, etc.), a pH adjuster (acetate, sodium citrate, cyclodextrin derivatives, sorbitan monolaurate, triethanolamine sodium acetate, triethanolamine oleate, etc.), an emulsifier, a solubilizer, an isotonic agent, an antiseptic, and a preservative.

There are no restriction on the dosage form and shape of the formulation. Examples of dosage forms include liquids, suspensions, injections, syrups, emulsions, jellies, tablets, pills, powders, fine granules, granules, capsules, topical agents, inhalants, nasal drops, eye drops, and suppositories.

The pharmaceutical composition according to the present invention contains an active ingredient in an amount necessary to achieve expected therapeutic effects (or preventive effects) (i.e., therapeutically effective dosage). Although the amount of an active ingredient in the pharmaceutical composition according to the present invention generally varies according to the dosage form, the amount of an active ingredient can be set within the range of, for example, about 0.001 wt % to about 99 wt % so that a desired dosage can be achieved.

Another aspect of the present invention relates to the use of the pharmaceutical composition. Typically, the pharmaceutical composition according to the present invention is used in the treatment, prevention, or amelioration of diseases or pathological conditions. The term “treatment” includes the alleviation (a change into a mild condition) of symptoms or concomitant symptoms characteristic of a target disease, and the prevention or delay of the exacerbation of symptoms. The term “prevention” refers to preventing or delaying the onset or development of a disease (disorder) or its symptoms, or reducing the risk of the onset or development of a disease (disorder) or its symptoms. The term “amelioration” means that a disease (disorder) or its symptoms are alleviated (made milder), improved, remitted, or cured (including being partially cured). Thus, treatment, prevention, and amelioration are in part overlapping concepts that are difficult to clearly distinguish from each other; there is little actual benefit in distinguishing them. In this specification, treatments aiming for prevention or amelioration are also included in the concept of the term “therapeutic method.”

The pharmaceutical composition according to the present invention is applied, for example, in the treatment of tumors. In a preferable embodiment, the pharmaceutical composition according to the present invention is used in the treatment of, in particular, malignant tumors (i.e., cancer) among tumors. In general, a cancer is referred to by the name of the organ or the name of the tissue from which the cancer has developed. The major cancers include tongue cancer, gum cancer, pharyngeal cancer, maxillary cancer, laryngeal cancer, salivary gland cancer, esophageal cancer, stomach cancer, small intestine cancer, large intestine cancer, rectal cancer, colon cancer, liver cancer, biliary tract cancer, gallbladder cancer, pancreas cancer, lung cancer, breast cancer, thyroid cancer, adrenal cancer, brain pituitary gland tumor, pineal tumor, uterus cancer, ovary cancer, vagina cancer, bladder cancer, kidney cancer, prostate cancer, urethral cancer, retinoblastoma, conjunctival cancer, neuroblastoma, glioma, glioblastoma, malignant melanoma (melanoma), medulloblastoma, leukemia, malignant lymphoma, testicular tumor, osteosarcoma, rhabdomyosarcoma, leiomyosarcoma, angiosarcoma, liposarcoma, chondrosarcoma, and Ewing's sarcoma. Cancers are further subdivided into, for example, upper, middle, or lower pharyngeal cancer, upper, middle, or lower esophageal cancer, gastric cardia cancer, gastropyloric cancer, cervical cancer, and endometrial cancer, according to the characteristics of the part of the organ where a cancer occurs. These are included in the concept of the term “cancer” without any limitation.

The cancer to be treated can be any cancer. Preferable examples of cancers to be treated include non-small cell lung cancer, small-cell lung cancer, breast cancer, stomach cancer, large intestine cancer, kidney cancer, head and neck cancer, malignant melanoma, Hodgkin's lymphoma, B-cell non-Hodgkin's lymphoma, mantle cell lymphoma, chronic lymphocytic leukemia, Philadelphia chromosome-positive acute lymphocytic leukemia, multiple myeloma, adult T-cell leukemia, peripheral T-cell lymphoma, cutaneous T-cell lymphoma, neuroblastoma, bladder cancer, ureteral cancer, angiosarcoma, rectal cancer, anus cancer, small intestine cancer, duodenal cancer, pancreas cancer, bile duct cancer, liver cancer, gallbladder cancer, esophageal cancer, GIST, malignant mesothelioma, thymic tumor, oral cancer, brain tumor, and sarcoma.

The use of the pharmaceutical composition according to the present invention is not limited to the treatment of tumors. For example, the pharmaceutical composition according to the present invention can also be applied to the treatment of various infectious diseases or various collagen diseases.

Examples of viruses that can cause infectious diseases include hepatitis A virus, hepatitis B virus, hepatitis C virus, human immunodeficiency virus (HIV), herpes simplex virus type 1 (HSV-1), herpes simplex virus type 2 (HSV-2), varicella-zoster virus (HHV-3), cytomegalovirus (HHV-5), human herpesvirus 6 (HHV-6), human herpesvirus 7 (HHV-7), Epstein-Barr virus (HHV-4), human herpesvirus 8 (HHV-8, also known as “Kaposi's sarcoma-associated herpesvirus” (KSHV)), influenza viruses, adenoviruses, norovirus, rotavirus, RS virus, various coronaviruses, measles virus, mumps virus, rhinovirus, dengue virus, papillomavirus, poliovirus, and rabies virus.

Similarly, examples of bacterial that can cause infectious diseases include Escherichia coli (E. coli), genus Shigella (S. dysenteriae, S. frexneri, S. sonnei, etc.), genus Salmonella (S. typh, S. paratyphi-A, S. schottmuelleri, S. typhimurium, S. enteritidis, etc.), genus Enterobacter (E. aerogenes, E. cloacae, etc.), genus Klebsiella (K. pneumoniae, K. oxytoca, etc.), genus Proteus (P. mirabilis, P. vulgaris, etc.), genus Yersinia (Y. pestis, Y. enterocolitica etc.), genus Vibrio (V. cholerae, V. parahaemolyticus, etc.), genus Haemophilus (H. influenzae, H. parainfluenzae, H. ducreyi, etc.), genus Pseudomonas (P. aeruginosa, P. cepacia, P. putida, etc.), genus Acinetobacter (A. calcoaceticus, A. baumannii, A. lwoffii, etc.), genus Legionella (L. pneumophila etc.), genus Bordetella (B. pertussis, B. parapertussis, B. bronchiseptica, etc.), genus Brucella (B. melitensis, B. abortus, B. suis, etc.), Francisella tularensis, genus Bacteroides (B. fragilis, B. melaninogenicus etc.), genus Neisseria (N. gonorrhoeae, N. meningitidis, etc.), genus Staphylococcus (S. aureus, S. epidermidis, S. saprophyticus etc.), genus Streptococcus (S. pyogenes, S. agalactiae, S. viridans, S. pneumoniae, etc.), genus Enterococcus (E. faecalis, E. faecium, E. avium, etc.), genus Bacillus (B. subtilis, B. anthracis, B. cereus, etc.), genus Clostridium (C. difficile, C. botulinum, C. perfringens, C. tetani, etc.), genus Corynebacterium (C. diphtheriae etc.), genus Mycobacterium (M. tuberculosis, M. bovis, M. leprae, M. avium, M. intracellulare, M. kansasii, M. ulcerans, etc.), Mycoplasma, genus Borrelia (B. recurrentis, B. burgdorferi etc.), Treponema pallidum, genus Campylobacter (C. coli, C. jejuni, C. fetus, etc.), genus Helicobacter (H. pylori, H. heilmannii, etc.), genus Rickettsia (R. prowazekil, R. mooseri, R. tsutsugamushi, etc.), genus Chlamydia (C. trachomatis, C. psittaci, etc.), and genus Listeria (L. monocytogenes etc.).

Examples of fungi that can cause infectious diseases include Candida (albicans, krusei, glabrata, tropicalis, etc.), Cryptococcus neoformans, Aspergillus (fumigatus, niger, etc.), genus Mucorales (Mucor, Absdia, Rhizopus), Sporothrix schenckii, Blastomyces dermatitidis, Paracoccidioides brasiliensis, Coccidioides immitis, and Histoplasma capsulatum.

Examples of parasites that can cause infectious diseases include Entamoeba histolytica parasite, Balantidium coli, Naegleria fowleri, Acanthamoeba species, Giardia lamblia, Cryptosporidium species, Pneumocystis carinii, Plasmodium vivax, Babesia microti, Trypanosoma brucei, Trypanosoma csruzi, Leishmania donovani, Toxoplasma gondii, and Ancylostoma braziliense.

Examples of collagen diseases include systemic lupus erythematosus, rheumatic fever, scleroderma, dermatomyositis, polymyositis, periarteritis nodosa, rheumatoid arthritis, Sjögren's syndrome, mixed connective tissue disease (MCTD), granulomatosis with polyangiitis (Wegener's granulomatosis), eosinophilic granulomatosis with polyangiitis (Churg-Strauss syndrome), microscopic polyangiitis, Takayasu's arteritis (aortitis syndrome), giant cell arteritis (temporal arteritis), polymyalgia rheumatica, eosinophilic fasciitis, adult Still's disease, ankylosing spondylitis, psoriatic arthritis, relapsing polychondritis, Behcet's disease, and sarcoidosis.

As described above, the pharmaceutical composition according to the present invention can be used in the treatment of various diseases or pathological conditions. The therapeutic method using the pharmaceutical composition according to the present invention includes the following steps (1) and (2): (1) administering the pharmaceutical composition according to the present invention to a treatment target to bind the target-specific conjugate according to the present invention to a target cell, and

(2) Irradiating the Target Cell with Near-Infrared Light.

In step (1), the pharmaceutical composition according to the present invention is administered to a treatment target. The route of administration can be selected, for example, according to the dosage form of the pharmaceutical composition and the treatment plan. Peroral administration and parenteral administration (intravenous, intraarterial, subcutaneous, intradermal, intramuscular, or intraperitoneal injection, transdermal, transnasal, or transmucosal administration, etc.) both can be used. These routes of administration are not mutually exclusive, and two or more of the routes may be used in combination (e.g., an intravenous injection may be performed simultaneously with an oral administration or after a predetermined period of time from an oral administration). The pharmaceutical composition may be administered locally (e.g., into a lesion or tumor) instead of systemically. The treatment target is typically a human, but may also be a non-human animal (e.g., non-human primates, domestic animals, pet animals, laboratory animals etc.). Specific examples include monkeys, chimpanzees, gorillas, orangutans, cows, pigs, goats, sheep, chickens, quails, dogs, cats, mice, rats, guinea pigs, and hamsters). A preferable target of application is humans.

The dosage of the pharmaceutical composition is determined so that the expected therapeutic effects are achieved. The symptoms, age, sex, and body weight of the patient are generally taken into consideration in determining a therapeutically effective dosage. A person skilled in the art can determine an appropriate dosage taking these factors into account. The dosage (the amount of an active ingredient (i.e., the target-specific conjugate)) is, for example, 0.1 to 1000 mg, 0.2 to 500 mg, 0.5 to 100 mg, or 1 to 20 mg, per 60 kg of body weight. A dosing schedule can also be created taking into consideration the patient's medical conditions or the duration of efficacy of the active ingredient.

After the target-specific conjugate (active ingredient) is bound to a target cell (i.e., the target-specific conjugate is bound to the surface of the target cell) by administering the pharmaceutical composition, the target cell is irradiated with near-infrared light (step (2)). Without being bound by theory, irradiation of near-infrared light induces selective necrotic cell death of the target cell (according to the principle of NIR-PIT). Along with the death of the target cell, the drug (payload) is released from the target-specific conjugate. The released payload diffuses across the surrounding area and acts on non-target cells around, causing damage to the cells according to the drug's efficacy. In this way, the target-specific effect based on the principle of NIR-PIT and the effect on the surrounding area by the drug are continuously produced to cause selective and extensive damage. For example, when applied to cancer treatment, the pharmaceutical composition can effectively attack the deep layers of cancerous tissue in addition to the surface layers, providing high therapeutic effects.

The irradiation with near-infrared light can be performed by using, for example, an LED, an LED laser, or filtered light beams. Devices for use in non-direct irradiation include, although not limited to, optically guided catheters, optically guided endoscope fibers, puncture irradiation fibers, optically guided blood vessel catheters, optically guided indwelling drain devices, implantable devices, patch devices, and bracelet devices. The conditions for irradiation of near-infrared light are not particularly limited as long as the damaging activity based on the principle of the NIR-PIT is achieved; the near-infrared light for use has a wavelength of, for example, 660 to 740 nm, preferably 670 to 720 nm, and more preferably 680 to 710 nm. The irradiation dose is, for example, at least 1 J cm⁻², at least 2 J cm⁻², at least 5 J cm⁻², at least 10 J cm⁻², at least 20 J cm⁻², at least 30 J cm⁻², at least 40 J cm⁻², at least 50 J cm⁻², at least 60 J cm⁻², at least 70 J cm⁻², at least 80 J cm⁻², at least 90 J cm⁻², or at least 100 J cm⁻². More specifically, the irradiation dose is, for example, 1 to 1000 J cm⁻², 2 to 500 J cm⁻², 5 to 300 J cm⁻², or 10 to 100 J cm⁻². The irradiation time is, for example, 5 seconds to 1 hour, 5 seconds to 30 minutes, or 5 seconds to 15 minutes. The irradiation time is preferably 10 seconds or more, still more preferably 1 minute or more, and yet more preferably 3 minutes or more. Although irradiation is not limited to this example, systemic administration of the pharmaceutical composition, for example, by an intravenous injection is followed by irradiation of near-infrared light performed at a point of time, for example, between 5 minutes and 48 hours, preferably between 10 minutes and 24 hours, and more preferably between 15 minutes and 12 hours after administration of the pharmaceutical composition. In local administration, it is preferable to set the interval between the administration of the pharmaceutical composition and the irradiation of near-infrared light shorter than in systemic administration.

Irradiation may be performed multiple times, instead of one time. For multiple-time irradiation, the interval is not particularly limited. For example, various irradiation schedules can be set, such as multiple-time irradiation on the same day with a predetermined interval (e.g., 5 minutes to 10 hours), irradiation on consecutive days, irradiation every other day or every few days, irradiation every week or every few weeks, irradiation every month or every few months, etc. The administration schedule of the pharmaceutical composition for multiple-time irradiation is not particularly limited. For example, when the interval between the first irradiation and the second irradiation is short, the pharmaceutical composition is typically administered only before the first irradiation. For another example, when a long time has passed since the last irradiation (e.g., when one day to several months have passed), the pharmaceutical composition may be administered again before irradiation is performed.

### EXAMPLES

### Development of Novel Photodynamic Pharmacotherapy

### 1. Preparation and Quality Confirmation of T-DM1 (Trastuzumab+N2′-Deacetyl-N2′-Maytansine)-IR700

### 1-1. Experimental Method

First, T-DM1-IR700 was synthesized. T-DM1 (1.0 mg, 6.6 nmol) and IR700 (66.8 μg, 34.2 nmol) were incubated together with 0.1 M Na₂HPO₄ (pH 8.5) at room temperature for 1 hour, and then passed through a Sephadex G50 column (PD-10, GE Healthcare) to recover a mixture solution (T-DM1-IR700 solution). After Coomassie staining, the absorbance (wavelength: 595 nm) was measured, and the concentration of T-DM1-IR700 (protein concentration) was determined. The concentration of IR700 was determined by measuring the absorbance (wavelength: 698 nm), and the number of fluorescent molecules bound to the antibody was confirmed. The mixture solution was subjected to SDS-PAGE to confirm the binding of the antibody to IR700. In the same manner, T-DM1-IR800 was also prepared. For the control in SDS-PAGE, diluted T-DM1 was used, and images were captured with a Pearl imager (LI-COR).

### 1-2. Results and Discussion

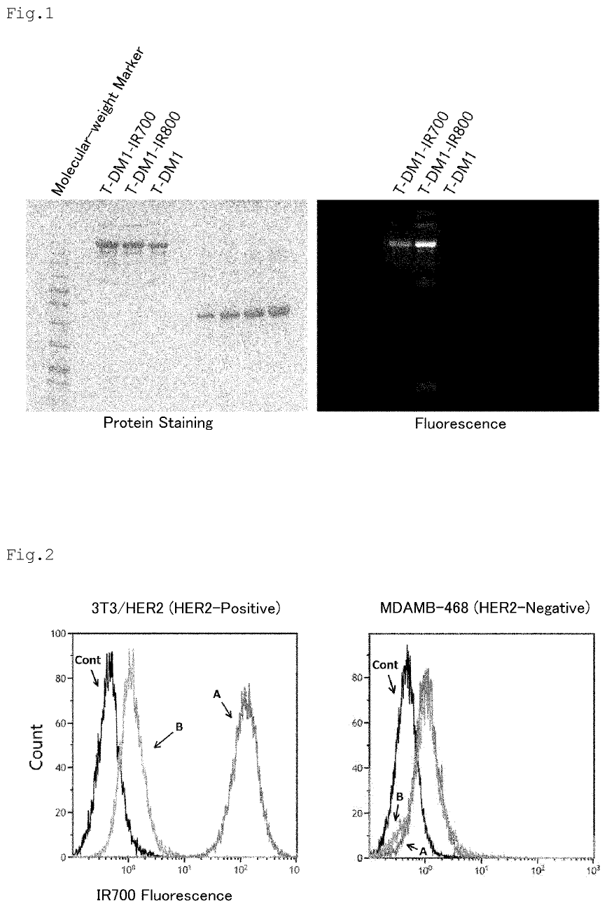

Fluorescence was only observed in T-DM1-IR700 (FIG. 1, right) at the height of the band of protein staining (FIG. 1, left), indicating that T-DM1 was bound (conjugated) to IR700.

### 2. Evaluation of Binding Properties of T-DM1-IR700 to HER2 Antigen

### 2-1. Experimental Method

3T3/HER2 (HER2-positive mouse fibroblast: HER2-positive) and MDAMB-468 Luc (human breast cancer cells constitutively expressing luciferase: HER2-negative) were individually seeded into a plate in an amount of 1×10⁵, and incubated with prepared T-DM1-IR700 at 37° C. for 6 hours, followed by measuring the fluorescence intensity by flow cytometry. To evaluate the specific binding ability of T-DM1-IR700 to HER2, 100 μg of trastuzumab (Tra) was first added to the cells to inhibit the binding of T-DM1-IR700 to the antigen, and then 10 μg of T-DM1-IR700 was administered, followed by measuring the fluorescence intensity.

### 2-2. Results and Discussion

The study with 3T3/HER2 showed enhanced fluorescence (FIG. 2, left), whereas the study with MDA-MB-468 did not show enhanced fluorescence (FIG. 2, right). This suggests that T-DM1-IR700 bonded only to HER2-positive cells. Additionally, the measurement of fluorescence after pre-administration of trastuzumab indicated a decrease in fluorescence in 3T3/HER2. Accordingly, T-DM1-IR700 is thought to have bonded specifically to the HER2 antigen.

### 3. Evaluation of Cell Proliferation Inhibition Activity

### 3-1. Experimental Method

3T3/HER2-luc cells and MDAMB-468-luc cells each in an amount of 1×10⁴ were individually seeded into a 24-well plate together with 1 ml of a medium. After 24 hours, the medium was replaced by a medium containing Tra-IR700 or T-DM1-IR700 of different concentrations. After 4 days, the luciferase activity was measured with a plate reader to assess the survival of HER2-negative cells.

### 3-2. Results and Discussion

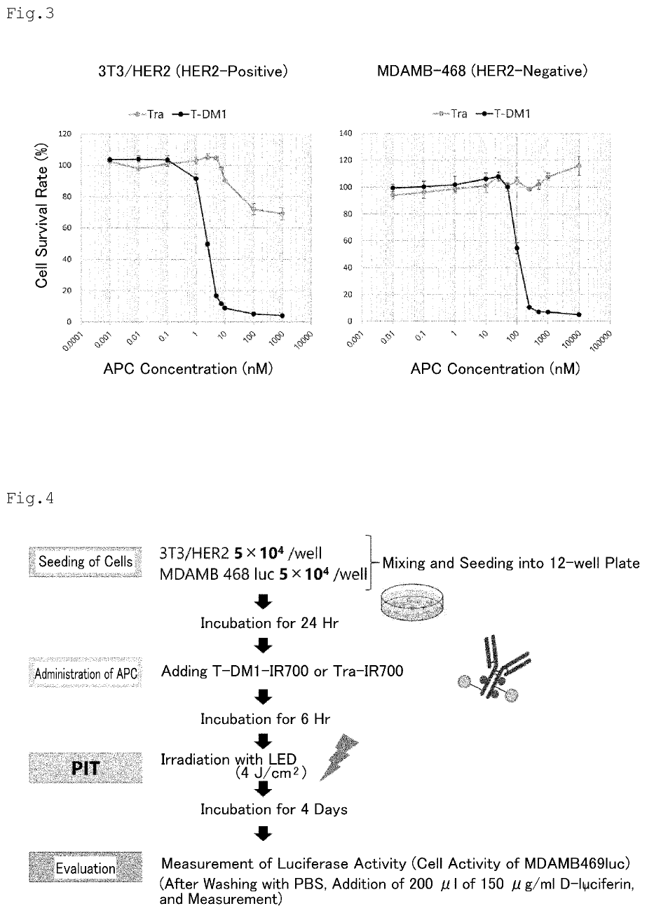

T-DM1-IR700 exhibited an antiproliferative effect on HER2-positive 3T3/HER2-luc cells (FIG. 3, left). The antiproliferative effect was also observed in HER2-negative MDAMB-468-luc cells when T-DM1-IR700 of high concentration was used (FIG. 3, right). This may be due to the effect of increased concentrations of the payload, which is non-specifically released from the monoclonal antibody portion of T-DM1, or due to the passive transport (non-specific uptake) of cell membranes caused by high concentrations. T-DM1-IR700 at normal concentrations at which side effects do not occur was confirmed to have no antiproliferative effect on MDAMB-468-luc cells. There was a 50-fold difference in IC₅₀ between the two types of cells. Whereas Tra-IR700 exhibited a mild antiproliferative effect on 3T3/HER2-luc cells (FIG. 3, left), Tra-IR700 did not exhibit an antiproliferative effect on MDAMB-468-luc (FIG. 3, right).

### 4. In Vitro NIR-PIT

### 4-1. Experimental Method (FIG. 4)

3T3/HER2 cells and MDA-MB-468-luc cells, each in an amount of 5×10⁴, were mixed and seeded into a 12-well plate for mixed culture. After 24-hour incubation, the supernatant was removed and replaced by a medium containing Tra-IR700 (10 μg/mL) or T-DM1-IR700 (1 μg/mL, 5 μg/mL, or 10 μg/mL), followed by additional incubation for 6 hours. Subsequently, the cells were washed with PBS twice and irradiated with near-infrared light (4 J cm²) by using an LED with an emission wavelength of 690 nm. After 4 days from treatment, the luciferase activity of the cells were measured, and the effect of NIR-PIT was evaluated. The cells were washed with PBS before the measurement of luciferase activity, and D-luciferin (150 μg/ml, 200 μl) was added to the plate, followed by quantitatively measuring the luminescence intensity of the luciferase with a plate reader.

### 4-2. Results and Discussion

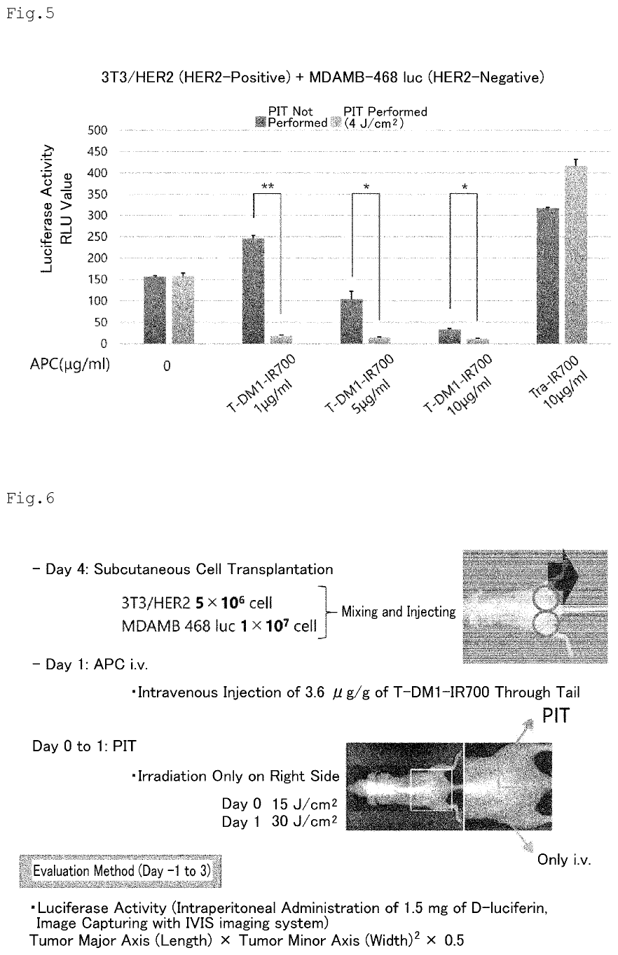

The amount of luminescence (RLU value) of luciferase activity was significantly decreased in the group for which NIR-PIT was performed with T-DM1-IR700, but not decreased in the control or in the group for which NIR-PIT was performed with Tra-IR700 (FIG. 5). Although a dose-dependent decrease in luciferase activity was observed with T-DM1-IR700 alone, the decrease in luciferase activity was more pronounced even at lower doses when NIR-PIT was performed. From these results, it is speculated that the photochemical reaction by NIR-PIT using T-DM1-IR700 promoted the release of the payload from the antibody, and thereby produced a cell-damaging effect (photo-bystander effect) even on HER2-negative MDA-MB-468 cells.

### 5. In Vivo NIR-PIT

### 5-1. Experimental Method (FIG. 6)

3T3/HER2 cells (5×10⁶) and MDAMB-468-luc cells (1×10⁷) were mixed in 150 μl of PBS, and transplanted subcutaneously into both dorsal buttocks of 8- to 10-week-old nude mice. The therapeutic effect of NIR-PIT was quantitatively evaluated by measuring the estimated tumor volume and the luciferase activity of tumors. The major axis and short axis of the tumors were measured, and the estimated tumor volume was calculated using “major axis×short axis²×½.” Mice with an estimated tumor volume greater than 100 mm³ were used for the experiment. The luciferase activity of tumors was measured by intraperitoneally administering D-luciferin (7.5 mg/mL, 200 ul), using an IVIS (registered trademark) imaging system. The unit of luminescence to be measured was radiance, and analysis was performed with Living Image Software (registered trademark). Cancer-bearing mice were divided into the following five groups: (1) PBS i.v., no light irradiation (control); (2) PBS i.v., with light irradiation, only right-side tumor; (3) Tra-IR700 (100 μg) i.v., with light irradiation, only right-side tumor; (4) T-DM1-IR700 (3.6 μg/g) i.v., no light irradiation; and (5) T-DM1-IR700 (3.6 μg/g) i.v., with light irradiation, only right-side tumor. After four days from subcutaneous implantation of cells, NIR-PIT was performed using a laser (15 J cm² after one day from i.v., 30 J cm² after 2 days from i.v.). After treatment, measurement was continued until the major axis of the tumors exceeded 20 mm.

### 5-2. Results and Discussion

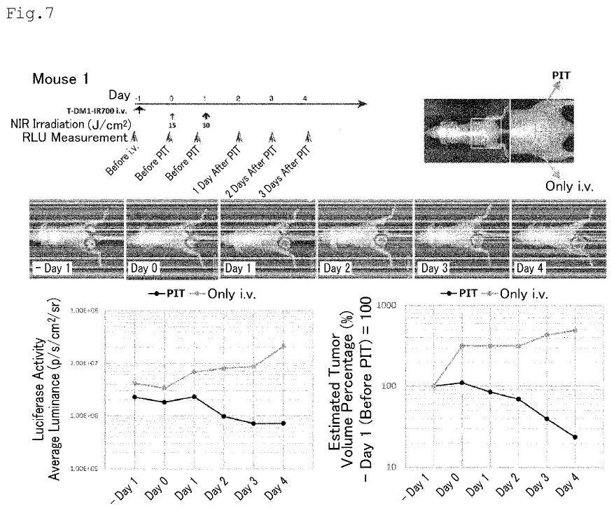

There was a significant difference between the group treated with NIR-PIT together with T-DM1-IR700 and the group treated with T-DM1-IR700 alone (FIG. 7). Regarding the luciferase activity of tumors, the RLU value of right-side tumors irradiated with a laser was decreased, whereas the RLU value of left-side tumors without irradiation was not decreased in mice to which T-DM1-IR700 was intravenously administered. The tumor volume on the right side was also smaller than that on the left side. Similarly to the results of the in vitro experiment, NIR-PIT using T-DM1-IR700 is thought to have had an effect not only on tumors in the HER2-positive cell portion but also on tumors in the HER2-negative cell portion (photo-bystander effect), suggesting a high antitumor effect.

### 6. Conclusion

NIR-PIT using a conjugate prepared by conjugating T-DM1 (ADC) and IR700 had a high antitumor effect. This innovative strategy, i.e., NIR-PIT using a conjugate of a target carrier (drug) and IR700, can address treatment resistance due to the heterogeneity of solid tumors, and allows for diffusion of a high-concentration drug locally in tumors and penetration of the drug deep into tumors, showing promise for high therapeutic effects. This strategy is a photodynamic pharmacotherapy usable not only in the field of tumors but also in a wide range of other diseases, such as infectious diseases and collagen diseases, for which antibody drugs are beginning to be used, and shows a great deal of potential application. The local therapeutic effect of the conjugate of a target carrier (drug) and IR700 on non-target cells around target cells is named “photo-bystander effect.”

### INDUSTRIAL APPLICABILITY

The target-specific conjugate (an assembly in which a drug and a substance sensitive to near-infrared light are linked to a molecule specifically bindable to a target molecule) according to the present invention exhibits a therapeutic effect (damaging activity) on target cells and a localized therapeutic effect (photo-bystander effect) on non-target cells around the target cells. This combined therapeutic effects will provide an effective treatment strategy for cases that have been difficult to treat. Additionally, because the target-specific conjugate is expected to provide higher therapeutic effects than conventional therapeutic methods, the target-specific conjugate will be used in or applied to various diseases and pathological conditions. The present invention, characterized by the photo-bystander effect, is an innovative technology set apart from ADCs or existing NIR-PIT.

The present invention is not limited in any way to the above description of embodiments and Examples of the invention. The present invention also includes various modifications of the invention to the extent that they can be easily conceived by those skilled in the art without departing from the scope of the claims. The research papers, published unexamined patent applications, and patent publications explicitly mentioned in this specification are incorporated by reference in their entirety.

1. A target-specific conjugate comprising
a molecule specifically bindable to a target molecule,
a drug, and
a substance sensitive to near-infrared light, wherein the drug and the substance sensitive to near-infrared light are linked to the specifically bindable molecule.

2. The target-specific conjugate according to claim 1, wherein the specifically bindable molecule is an antibody or an antigen-binding antibody fragment.

3. The target-specific conjugate according to claim 1, wherein the target molecule is a cell surface protein.

4. The target-specific conjugate according to claim 3, wherein the cell surface protein is a tumor-specific protein.

5. The target-specific conjugate according to claim 4, wherein the tumor-specific protein is HER1, HER2, HER3, CD3, CD19, CD20, CD25, CD26, CD33, CD44, CD52, PDL-1, CTLA-4, EpCAM, GD2, VEGFR, VEGFR2, CCR4, PMSA, mesothelin, GPC3, CEA, MUC1, c-KIT, DLL-3, PDPN, GPR85, GPR78, Cadherin3, Trop-2, B7-H3, or an ephrin receptor.

6. The target-specific conjugate according to claim 1, wherein the drug is a cell-damaging drug.

7. The target-specific conjugate according to claim 6, wherein the cell-damaging drug is one, or two or more drugs selected from the group consisting of alkylating agents, platinum-based drugs, antimetabolites, antitumor antibiotics, microtubule polymerization inhibitors, microtubule depolymerization inhibitors, topoisomerase inhibitors, vegetable alkaloids, hormonal agents, and bacteria-derived toxins.

8. The target-specific conjugate according to claim 4, wherein the drug is an anticancer agent.

9. The target-specific conjugate according to claim 1, wherein the substance sensitive to near-infrared light is a phthalocyanine dye.

10. The target-specific conjugate according to claim 9, wherein the phthalocyanine dye is IR700.

11. A pharmaceutical composition comprising the target-specific conjugate of claim 1.

12. The pharmaceutical composition according to claim 11, which is for use in the treatment or prevention of a cancer.

13. The pharmaceutical composition according to claim 12, wherein the cancer is non-small cell lung cancer, small-cell lung cancer, breast cancer, stomach cancer, large intestine cancer, kidney cancer, head and neck cancer, malignant melanoma, Hodgkin's lymphoma, B-cell non-Hodgkin's lymphoma, mantle cell lymphoma, chronic lymphocytic leukemia, Philadelphia chromosome-positive acute lymphocytic leukemia, multiple myeloma, adult T-cell leukemia, peripheral T-cell lymphoma, cutaneous T-cell lymphoma, neuroblastoma, bladder cancer, ureteral cancer, angiosarcoma, rectal cancer, anus cancer, small intestine cancer, duodenal cancer, pancreas cancer, bile duct cancer, liver cancer, gallbladder cancer, esophageal cancer, GIST, malignant mesothelioma, thymic tumor, oral cancer, or brain tumor.

14. A therapeutic method comprising the following steps (1) and (2):
(1) administering the pharmaceutical composition of claim 11 to a treatment target to bind the target-specific conjugate to a target cell, and
(2) irradiating the target cell with near-infrared light.

15. The therapeutic method according to claim 14, wherein the near-infrared light has a wavelength of 660 to 740 nm.

16. The therapeutic method according to claim 14, wherein the near-infrared light has a wavelength of 670 to 720 nm.

17. The therapeutic method according to claim 14, wherein an irradiation dose of the near-infrared light is 1 J cm⁻² or more.

18. The therapeutic method according to claim 14, wherein an irradiation dose of the near-infrared light is 2 J cm⁻² to 500 J cm⁻².

19. The therapeutic method according to claim 14, wherein an irradiation dose of the near-infrared light is 5 J cm⁻² to 300 J cm⁻².

20. The therapeutic method according to claim 14, wherein after the irradiation with the near-infrared light induces necrotic cell death, the drug linked to the target-specific conjugate is diffused to cause damage to surrounding cells.
